(19) United States
(12) Patent Application Publication
(10) Pub. No.: US 2026/0217635 A1
(43) Pub. Date: Jul. 30, 2026

(54) SYSTEM AND METHOD FOR SEPARATION OF NON-IDEAL STREAMS

(71) Applicant: SAUDI ARABIAN OIL COMPANY, Dhahran (SA)

(72) Inventors: Ali JAWAD, Dhahran (SA); Mohammad Abdur RAKIB, Dhahran (SA); Ahmad KHAN, Dhahran (SA); Mohnnad ALABSI, Dhahran (SA); Vishvedeep BHAT, Dhahran (SA)

(73) Assignee: SAUDI ARABIAN OIL COMPANY, Dhahran (SA)

(21) Appl. No.: 19/040,468

(22) Filed: Jan. 29, 2025

(51) Int. Cl. C07C 41/38; B01D 3/14; B01D 3/40; B01D 53/00; B01D 53/047; C01B 3/06; C07C 7/00; C07C 29/86

(52) U.S. Cl. CPC C07C 41/38; B01D 3/143; B01D 3/40; B01D 53/002; B01D 53/047; C01B 3/06; C07C 7/005; C07C 29/86; B01D 2256/16

(57) ABSTRACT

Processes for separating components from a reactor stream may comprise the steps of: separating a first vapor recovery stream and a liquid recovery stream from the reactor stream; forming a hydrogen stream, a heavier hydrocarbons fraction, alcohols fraction, and water fraction stream, and a carbon dioxide stream from the first vapor recovery stream; and forming an aqueous rich stream comprising about a 2:1 ratio or greater of smaller alcohols to larger alcohols and a hydrocarbon rich stream comprising about a 2:1 ratio or greater of larger alcohols to smaller alcohols from the liquids recovery stream.

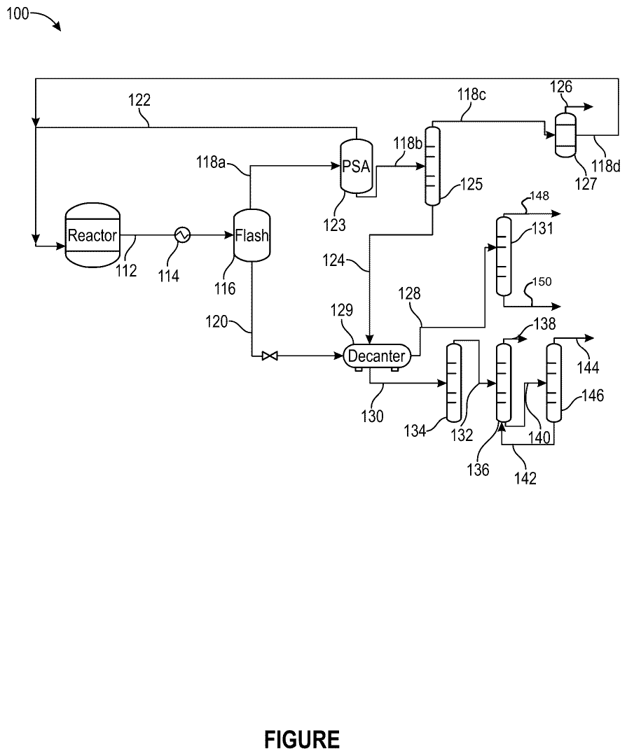

### FIELD

The present disclosure generally relates to methods and systems for separating reactor streams comprising hydrocarbons, alcohols, water, and non-condensable gases, and more particularly, to methods for separating the components of reactor streams.

### BACKGROUND

Chemical manufacturing facilities create fuels, chemical feedstocks, and other valuable chemicals through various chemical reactions. Example reactions include etherification of olefins and Fischer-Tropsch (FT) reactions. These reactions can be performed via multiple reactors or in a single reactor. When these reactions are performed in a single reactor, they produce reactor streams comprising hydrocarbons, oxygenates (e.g., alcohols, ethers), water, and non-condensable gases.

The separation of these reactor streams into separate hydrocarbon, oxygenate, water, and non-condensable gas streams involves several challenges. These reactor streams are azeotropes, meaning that two or more components boil at the same temperature and hinder the complete separation of the mixed product streams through distillation. Additionally, similar boiling points of the various components in the mixed product streams further frustrates separation of the mixed product streams. Phase separation issues also complicate the process. While water and hydrocarbons typically form immiscible phases, oxygenates can act as solvents, leading to partial miscibility and creating emulsion-like mixtures that are difficult to separate through decantation or other physical methods. This typically requires the use of coalescers, demulsifiers, or additional chemical additives to break the reactor streams into its various components. The energy-intensive nature of these separation processes further adds to the complexity. Multiple stages of distillation, dehydration (such as using molecular sieves for water removal), and other processes are often required, driving up operational costs. Maintaining high purity levels for certain applications, such as fuels or chemical feedstocks, imposes additional demands on the separation processes.

### SUMMARY

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

In one or more aspects, the present disclosure provides a method for separating components from a reactor stream comprising the steps of: separating a first vapor recovery stream and a liquid recovery stream from the reactor stream; forming a hydrogen stream, a heavier hydrocarbons fraction, alcohols fraction, and water fraction stream, and a carbon dioxide stream from the first vapor recovery stream; and forming an aqueous rich stream comprising about a 2:1 ratio or greater of smaller alcohols to larger alcohols and a hydrocarbon rich stream comprising about a 2:1 ratio or greater of larger alcohols to smaller alcohols from the liquids recovery stream.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The FIGURE shows a schematic diagram of an embodiment of a system and method of the present disclosure.

### DETAILED DESCRIPTION

The present disclosure generally relates to methods and systems for separating reactor streams comprising hydrocarbons, alcohols, water, and non-condensable gases, and more particularly, to methods for separating the components of reactor streams. The methods and systems of the present disclosure allow for the phase separation, product purification, and recovery of reactor streams comprising hydrocarbons, alcohols, water, and non-condensable gases. The methods and systems enable the recycling of hydrogen and carbon dioxide for reuse in chemical manufacturing processes. The methods and systems may allow for greater than 95% main and by-product recovery with greater than 97% purity.

The present disclosure provides a process for separating components from a reactor stream comprising the steps of: separating a first vapor recovery stream and a liquid recovery stream from the reactor stream; forming a hydrogen stream, a heavier hydrocarbons fraction, alcohols fraction, and water fraction stream, and a carbon dioxide stream from the first vapor recovery stream; and forming an aqueous rich stream comprising about a 2:1 ratio or greater of smaller alcohols to larger alcohols and a hydrocarbon rich stream comprising about a 2:1 ratio or greater of larger alcohols to smaller alcohols from the liquids recovery stream.

Turning to the FIGURE, the FIGURE shows chemical processing system 100. Chemical processing system 100 may include reactor stream 112. Reactor stream 112 may be produced by any suitable process involving a reactor such as a gas-to-liquids process (e.g., Fischer-Tropsch reactions), etherification of olefins, the like, or combinations thereof. Reactor stream 112 may include hydrocarbons, alcohols, ethers, other oxygenates, water, non-condensable gases, or combinations thereof.

Hydrocarbons may include light hydrocarbons and heavier hydrocarbons. Light hydrocarbons may include C1 to C4 hydrocarbons. Heavier hydrocarbons may include C5 and larger hydrocarbons, e.g., C5 to C20 hydrocarbons. Oxygenates may include alcohols, aldehydes, ketones, ethers, esters, or combinations thereof. The oxygenates may be of various sizes, including C1 to C10 oxygenates. Oxygenates may include methanol, ethanol, propanol, butanol, pentanol, hexanol, heptanol, octanol, or combinations thereof. Smaller alcohols may include methanol and ethanol. Larger alcohols may include alcohols larger than ethanol (e.g., propanol, butanol, pentanol and so forth). Non-condensable gases may include carbon dioxide, carbon monoxide, hydrogen gas, light hydrocarbons, or combinations thereof.

Reactor stream 112 may be in a vapor phase and may be condensed via optional condenser 114 to produce a vapor-liquid phase mixture. Before interacting with condenser 114, reactor stream 112 may be at a temperature of about 300° C. to about 400° C., including all values and subsets therebetween, and at a pressure of about 20 bar to about 40 bar, including all values and subsets therebetween. Condenser 114 may cool reactor stream 112 with water or another coolant to condense the vapor phase.

Reactor stream 112 may be separated into first vapor recovery stream 118a and liquid recovery stream 120. The separation of reactor stream 112 into first vapor recovery stream 118a and liquid recovery stream 120 may be performed by flash drum 116 or any apparatus capable of separating components based on their boiling points.

First vapor recovery stream 118a may include carbon dioxide, carbon monoxide, hydrogen gas, hydrocarbons, water, alcohols, or combinations thereof. First vapor recovery stream 118a may be at a temperature of about 25° C. to about 45° C., including all values and subsets therebetween, and a pressure of about 20 bar to about 40 bar, including all values and subsets therebetween. First vapor recovery stream may be separated into hydrogen, heavier hydrocarbons, and carbon dioxide through chemical processing system 100.

First vapor recovery stream 118a may be separated into hydrogen stream 122 and second vapor recovery stream 118b. Separation of first vapor recovery stream 118a into hydrogen stream 122 and second vapor recovery stream 118b may be performed by utilizing pressure swing adsorption (PSA) bed 123 or any other suitable method/apparatus capable of separating hydrogen from a stream. Optionally, hydrogen stream 122 may be fed back to the reactor that produced reactor stream 112. Second vapor recovery stream 118b may be at a temperature of about 25° C. to about 45° C., including all values and subsets therebetween, and a pressure of about 20 bar to about 40 bar, including all values and subsets therebetween. Hydrogen stream 122 may be at a temperature of about 25° C. to about 45° C., including all values and subsets therebetween, and a pressure of about 20 bar to about 40 bar, including all values and subsets therebetween.

The second vapor recovery stream 118b may be separated into third vapor recovery stream 118c and water fraction stream 124 that comprises a heavier hydrocarbons fraction, alcohols fraction, and water fraction. Third vapor recovery stream 118c may include carbon dioxide, lighter hydrocarbons, or combinations thereof. Separation of second vapor recovery stream 118b into third vapor recovery stream 118c and heavier hydrocarbons fraction, alcohols fraction, and water fraction stream 124 may be performed by cryogenic distillation column 125 or any suitable method or apparatus capable of separating components of a stream based on their relative volatilities. Third vapor recovery stream 118c may be at a temperature of about −7° C. to about 27° C., including all values and subsets therebetween, and a pressure of about 10 bar to about 30 bar, including all values and subsets therebetween. Heavier hydrocarbons fraction, alcohols fraction, and water fraction stream 124 may be at a temperature of about −8° C. to about 18° C., including all values and subsets therebetween, and a pressure of about 10 bar to about 30 bar, including all values and subsets therebetween. Optionally, heavier hydrocarbons fraction, alcohols fraction, and water fraction stream 124 may be combined with liquid recovery stream 120.

In one or more embodiments, third vapor recovery stream 118c may be separated into fourth vapor recovery stream 118d and light hydrocarbons stream 126. Fourth vapor recovery stream 118d may include carbon dioxide. Fourth vapor recovery stream 118d may be fed back to the reactor that produced reactor stream 112, if desired. Light hydrocarbons stream 126 may include C1 to C4 hydrocarbons. The separation of third vapor recovery stream 118c into fourth vapor recovery stream 118d and light hydrocarbons stream 126 may be performed by carbon recovery unit 127, which may perform absorption (physical or chemical), adsorption, or use membranes for separation. In one or more embodiments, third vapor recovery stream 118c may be combined with steam to undergo steam reformation to produce fourth vapor recovery stream 118d where fourth vapor recovery stream 118d includes syngas.

Liquid recovery stream 120 may include light hydrocarbons, heavier hydrocarbons, water, alcohols, or combinations thereof. Water may be the most prevalent component of liquid recovery stream 120. Of the hydrocarbons in liquid recovery stream 120, in some embodiments, there may be a majority of heavier hydrocarbons versus lighter hydrocarbons. Of the alcohols in liquid recovery stream 120, there may be a majority of smaller alcohols versus larger alcohols. Liquid recovery stream 120 may be at a temperature of about 25° C. to about 45° C., including all values and subsets therebetween, and a pressure of about 1 bar to about 10 bar, including all values and subsets therebetween.

Liquid recovery stream 120 may be separated into hydrocarbon rich stream 128 and aqueous rich stream 130. As discussed above, liquid recovery stream 120 may include alcohols. Without intending to be bound by theory, the larger alcohols in liquid recovery stream 120 may primarily associate with the hydrocarbons and the smaller alcohols may primarily associate with the water. Thus, in some instances, aqueous rich stream 130 may include about a 2:1 ratio or greater of smaller alcohols to larger alcohols and hydrocarbon rich stream 128 may include about a 2:1 ratio or greater of larger alcohols to smaller alcohols. The separation of liquid recovery stream 120 into hydrocarbon rich stream 128 and aqueous rich stream 130 may be performed by decanter 129 or other apparatus capable of separating aqueous rich and hydrocarbon rich components from a stream. Hydrocarbon rich stream 128 may be at a temperature of about 150° C. to about 175° C., including all values and subsets therebetween, and a pressure of about 1 bar to about 15 bar, including all values and subsets therebetween.

Hydrocarbon rich stream 128 may be separated into a light hydrocarbons and larger alcohols stream 148 and a heavier hydrocarbons stream 150 by fractional distillation via hydrocarbon fractional distillation column 131. The larger alcohols may be removed at the top of hydrocarbon fractional distillation column 131 along with the light hydrocarbons. The larger alcohols and light hydrocarbons may be separated via adsorbent filtration or additives.

Aqueous rich stream 130 may be separated into water and water-alcohol stream 132. The separation of aqueous rich stream 130 into water and water-alcohol stream 132 may be performed by aqueous rich stream fractional distillation column 134.

Water-alcohol stream 132 may be combined with an entrainer and then extractive distillation performed via extractive distillation column 136 to produce vaporized alcohol stream 138 and water-entrainer stream 140. Suitable entrainers include compounds that alter the relative volatility of the azeotropic components. Example entrainers may include ethylene glycol, benzene, cyclohexane, or the like.

Water-entrainer stream 140 may be stripped into entrainer stream 142 and water stream 144 by stripping column 146. Entrainer stream 142 may be recombined with water-alcohol stream 132 and recycled back to extractive distillation column 136 for reuse in the extractive distillation process. Aqueous rich stream 130, water-alcohol stream 132, and vaporized alcohol stream 138 may each have the same or different temperatures and/or pressures, which may be about 25° C. to about 60° C., including all values and subsets therebetween, and a pressure of about 1 bar to about 10 bar, including all values and subsets therebetween.

In one or more embodiments, each stream may have a temperature, pressure, and weight percentage composition as shown in Table 1 below. Each stream element in the table corresponds to a stream as discussed herein. For example, element 122 in Table 1 corresponds to hydrogen stream 122. As shown by below Table 1, hydrogen stream 122 may have a temperature of about 35° C., be at about 30 bar, and may comprise about 99 wt % hydrogen gas.

TABLE 1

| | Stream | | | | | | | | | | |
|---|---|---|---|---|---|---|---|---|---|---|---|
| | 112 | 118a | 118b | 122 | 118c | 124 | 120 | 128 | 130 | 132 | 138 |
| T [° C.] | 350 | 35 | 35 | 35 | −17 | 8 | 35 | 162 | 35 | 48 | 48 |
| P [Bar] | 30 | 30 | 30 | 30 | 20 | 20 | 1 | 10 | 1 | 1 | 1 |
| wt % | | | | | | | | | | | |
| CO₂ | 44 | 77 | 89 | | 90 | | | | | | |
| CO | 2 | | | | | | | | | | |
| H₂ | 8 | 14 | 11 | 99 | 10 | | | | | | |
| C₁-C₄ HC | 11 | 9 | | <1 | | | 20 | <1 | | | |
| C₅₊ HC | | | | | | 70 | | 99 | | | |
| H₂O | 32 | | | | | 22 | 76 | | 94 | 85 | 2 |
| Alcohols | 2 | | | | | 8 | 4 | <1 | 6 | 15 | 98 |

### NON-LIMITING CLAUSES

The present disclosure is further directed to the following non-limiting clauses:
Clause 1: A process for separating components from a reactor stream comprising the steps of: separating a first vapor recovery stream and a liquid recovery stream from the reactor stream; forming a hydrogen stream, a heavier hydrocarbons fraction, alcohols fraction, and water fraction stream, and a carbon dioxide stream from the first vapor recovery stream; and forming an aqueous rich stream comprising about a 2:1 ratio or greater of smaller alcohols to larger alcohols and a hydrocarbon rich stream comprising about a 2:1 ratio or greater of larger alcohols to smaller alcohols from the liquids recovery stream.
Clause 2: The process of clause 1, further comprising before separating the first vapor recovery stream and the liquid recovery stream from the reactor stream, condensing the reactor stream.
Clause 3: The process of clause 1 or 2, wherein the smaller alcohols comprise methanol and ethanol and the larger alcohols comprise alcohols larger than ethanol.
Clause 4: The process of any of clauses 1-3, wherein the reactor stream comprises hydrocarbons, water, alcohols, and non-condensable gases.
Clause 5: The process of any of clauses 1-4, wherein the non-condensable gases comprise gases selected from the group consisting of carbon dioxide, carbon monoxide, hydrogen gas, light hydrocarbons, and combinations thereof.
Clause 6: The process of any of clauses 1-5, wherein the reactor stream is produced by a process selected from the group consisting of etherification of olefins, Fischer-Tropsch reactions, and combinations thereof.
Clause 7: The process of any of clauses 1-6, wherein water is the most prevalent component of the liquid recovery stream.
Clause 8: The process of any of clauses 1-7, wherein the first vapor recovery stream comprises carbon dioxide, hydrogen gas, light hydrocarbons and heavier hydrocarbons.
Clause 9: The process of any of clauses 1-8, wherein the liquid recovery stream comprises heavier hydrocarbons, water, and alcohols.
Clause 10: The process of any of clauses 1-9, further comprising separating the hydrocarbon rich stream into a light hydrocarbons and larger alcohols stream and a heavier hydrocarbons stream.
Clause 11: The process of any of clauses 1-10, further comprising separating the aqueous rich stream into a water and a water-alcohol stream.
Clause 12: The process of any of clauses 1-11, further comprising combining the water-alcohol stream with an entrainer and separating an alcohol stream from the water-entrainer stream.
Clause 13: The process of any of clauses 1-12, further comprising stripping the water-entrainer stream to produce a water stream and an entrainer stream and recombining the entrainer stream with the water-alcohol stream.
Clause 14: The process of any of clauses 1-13, further comprising feeding the hydrogen stream to a reactor.
Clause 15: The process of any of clauses 1-14, further comprising combining the heavier hydrocarbons, alcohols, and water stream with the liquid recovery stream.
Clause 16: The process of any of clauses 1-15, further comprising feeding the carbon dioxide stream to a reactor.
Clause 17: The process of any of clauses 1-16, further comprising combining the carbon dioxide stream with steam and performing steam reformation.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component or composition not expressly recited or disclosed herein. Any method may lack any step not recited or disclosed herein. Likewise, the term “comprising” is considered synonymous with the term “including.” Whenever a method, composition, element, or group of elements is preceded with the transitional phrase “comprising,” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related, and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned, as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

What is claimed is the following:

1. A process for separating components from a reactor stream comprising the steps of:
separating a first vapor recovery stream and a liquid recovery stream from the reactor stream;
forming a hydrogen stream, a heavier hydrocarbons fraction, alcohols fraction, and water fraction stream, and a carbon dioxide stream from the first vapor recovery stream; and
forming an aqueous rich stream comprising about a 2:1 ratio or greater of smaller alcohols to larger alcohols and a hydrocarbon rich stream comprising about a 2:1 ratio or greater of larger alcohols to smaller alcohols from the liquids recovery stream.

2. The process of claim 1, further comprising before separating the first vapor recovery stream and the liquid recovery stream from the reactor stream, condensing the reactor stream.

3. The process of claim 1, wherein the smaller alcohols comprise methanol and ethanol and the larger alcohols comprise alcohols larger than ethanol.

4. The process of claim 1, wherein the reactor stream comprises hydrocarbons, water, alcohols, and non-condensable gases.

5. The process of claim 4, wherein the non-condensable gases comprise gases selected from the group consisting of carbon dioxide, carbon monoxide, hydrogen gas, light hydrocarbons, and combinations thereof.

6. The process of claim 1, wherein the reactor stream is produced by a process selected from the group consisting of etherification of olefins, Fischer-Tropsch reactions, and combinations thereof.

7. The process of claim 1, wherein water is the most prevalent component of the liquid recovery stream.

8. The process of claim 1, wherein the first vapor recovery stream comprises carbon dioxide, hydrogen gas, light hydrocarbons and heavier hydrocarbons.

9. The process of claim 1, wherein the liquid recovery stream comprises heavier hydrocarbons, water, and alcohols.

10. The process of claim 1, further comprising separating the hydrocarbon rich stream into a light hydrocarbons and larger alcohols stream and a heavier hydrocarbons stream.

11. The process of claim 1, further comprising separating the aqueous rich stream into a water and a water-alcohol stream.

12. The process of claim 11, further comprising combining the water-alcohol stream with an entrainer and separating an alcohol stream from the water-entrainer stream.

13. The process of claim 12, further comprising stripping the water-entrainer stream to produce a water stream and an entrainer stream and recombining the entrainer stream with the water-alcohol stream.

14. The process of claim 1, further comprising feeding the hydrogen stream to a reactor.

15. The process of claim 1, further comprising combining the heavier hydrocarbons, alcohols, and water stream with the liquid recovery stream.

16. The process of claim 1, further comprising feeding the carbon dioxide stream to a reactor.

17. The process of claim 1, further comprising combining the carbon dioxide stream with steam and performing steam reformation.
